(19) United States
(12) Patent Application Publication
(10) Pub. No.: US 2005/0197657 A1
(43) Pub. Date: Sep. 8, 2005

(54) Thermokeratoplasty system with a regulated power generator

(76) Inventors: Paul R. Goth, Bristol, ME (US); William J. Bowers, Highlands Ranch, CO (US); Dorin Panescu, San Jose, CA (US)

Correspondence Address: IRELL & MANELLA LLP, 840 NEWPORT CENTER DRIVE, SUITE 400, NEWPORT BEACH, CA, 92660, US

(21) Appl. No.: 10/792,152

(22) Filed: Mar. 2, 2004

(51) Int. Cl. A61B 18/14

(52) U.S. Cl. 606/41

(57) ABSTRACT

An apparatus that provides energy to a probe in contact with a cornea to perform a medical procedure. The apparatus includes a circuit that supplies energy to the probe and a regulator that regulates the delivery of energy during the medical procedure. The apparatus may include a sensing circuit that senses a change in a physiology of the cornea. The regulator can vary the energy delivered to the cornea in accordance with changes in the cornea physiology. For example, a waveform of tissue impedance may be determined and compared to a desired waveform. Deviations from the desired waveform may cause the regulator to increase or decrease the power applied to the cornea.

### CROSS-REFERENCE TO RELATED APPLICATIONS

This application is a continuation-in-part of Application No. 819,561, filed on Mar. 27, 2001, pending.

### BACKGROUND OF THE INVENTION

1. Field of the Invention

The present invention relates to a thermokeratoplasty system that is used to reshape a cornea.

2. Prior Art

Techniques for correcting vision have included reshaping the cornea of the eye. For example, myopic conditions can be corrected by cutting a number of small incisions in the corneal membrane. The incisions allow the corneal membrane to relax and increase the radius of the cornea. The incisions are typically created with either a laser or a precision knife. The procedure for creating incisions to correct myopic defects is commonly referred to as radial keratotomy and is well known in the art.

Radial keratotomy techniques generally make incisions that penetrate approximately 95% of the cornea. Penetrating the cornea to such a depth increases the risk of puncturing the Descemets membrane and the endothelium layer, and creating permanent damage to the eye. Additionally, light entering the cornea at the incision sight is refracted by the incision scar and produces a glaring effect in the visual field. The glare effect of the scar produces impaired night vision for the patient.

The techniques of radial keratotomy are only effective in correcting myopia. Radial keratotomy cannot be used to correct an eye condition such as hyperopia. Additionally, keratotomy has limited use in reducing or correcting an astigmatism. The cornea of a patient with hyperopia is relatively flat (large spherical radius). A flat cornea creates a lens system which does not correctly focus the viewed image onto the retina of the eye. Hyperopia can be corrected by reshaping the eye to decrease the spherical radius of the cornea. It has been found that hyperopia can be corrected by heating and denaturing local regions of the cornea. The denatured tissue contracts and changes the shape of the cornea and corrects the optical characteristics of the eye. The procedure of heating the corneal membrane to correct a patient's vision is commonly referred to as thermokeratoplasty.

U.S. Pat. No. 4,461,294 issued to Baron; U.S. Pat. No. 4,976,709 issued to Sand and PCT Publication WO 90/12618, all disclose thermokeratoplasty techniques which utilize a laser to heat the cornea. The energy of the laser generates localized heat within the corneal stroma through photonic absorption. The heated areas of the stroma then shrink to change the shape of the eye.

Although effective in reshaping the eye, the laser based systems of the Baron, Sand and PCT references are relatively expensive to produce, have a non-uniform thermal conduction profile, are not self limiting, are susceptible to providing too much heat to the eye, may induce astigmatism and produce excessive adjacent tissue damage, and require long term stabilization of the eye. Expensive laser systems increase the cost of the procedure and are economically impractical to gain widespread market acceptance and use.

Additionally, laser thermokeratoplasty techniques non-uniformly shrink the stroma without shrinking the Bowmans layer. Shrinking the stroma without a corresponding shrinkage of the Bowmans layer, creates a mechanical strain in the cornea. The mechanical strain may produce an undesirable reshaping of the cornea and probable regression of the visual acuity correction as the corneal lesion heals. Laser techniques may also perforate Bowmans layer and leave a leucoma within the visual field of the eye.

U.S. Pat. Nos. 4,326,529 and 4,381,007 issued to Doss et al, disclose electrodes that are used to heat large areas of the cornea to correct for myopia. The electrode is located within a sleeve that suspends the electrode tip from the surface of the eye. An isotropic saline solution is irrigated through the electrode and aspirated through a channel formed between the outer surface of the electrode and the inner surface of the sleeve. The saline solution provides an electrically conductive medium between the electrode and the corneal membrane. The current from the electrode heats the outer layers of the cornea. Heating the outer eye tissue causes the cornea to shrink into a new radial shape. The saline solution also functions as a coolant which cools the outer epithelium layer.

The saline solution of the Doss device spreads the current of the electrode over a relatively large area of the cornea. Consequently, thermokeratoplasty techniques using the Doss device are limited to reshaped corneas with relatively large and undesirable denatured areas within the visual axis of the eye. The electrode device of the Doss system is also relatively complex and cumbersome to use.

“A Technique for the Selective Heating of Corneal Stroma” Doss et al., Contact & Intraoccular Lens Medical Jrl., Vol. 6, No. 1, pp. 13-17, January-March, 1980, discusses a procedure wherein the circulating saline electrode (CSE) of the Doss patent was used to heat a pig cornea. The electrode provided 30 volts r.m.s. for 4 seconds. The results showed that the stroma was heated to 70° C. and the Bowman's membrane was heated 45° C., a temperature below the 50-55° C. required to shrink the cornea without regression.

“The Need For Prompt Prospective Investigation” McDonnell, Refractive & Corneal Surgery, Vol. 5, January/February, 1989 discusses the merits of corneal reshaping by thermokeratoplasty techniques. The article discusses a procedure wherein a stromal collagen was heated by radio frequency waves to correct for a keratoconus condition. As the article reports, the patient had an initial profound flattening of the eye followed by significant regression within weeks of the procedure.

“Regression of Effect Following Radial Thermokeratoplasty in Humans” Feldman et al., Refractive and Corneal Surgery, Vol. 5, September/October, 1989, discusses another thermokeratoplasty technique for correcting hyperopia. Feldman inserted a probe into four different locations of the cornea. The probe was heated to 600° C. and was inserted into the cornea for 0.3 seconds. Like the procedure discussed in the McDonnell article, the Feldman technique initially reduced hyperopia, but the patients had a significant regression within 9 months of the procedure.

Refractec, Inc. of Irvine Calif., the assignee of the present application, has developed a system to correct hyperopia with a thermokeratoplasty probe that is connected to a console. The probe includes a tip that is inserted into the stroma layer of a cornea. Electrical current provided by the console flows through the eye to denature the collagen tissue within the stroma. The process of inserting the probe tip and applying electrical current can be repeated in a circular pattern about the cornea. The denatured tissue will change the refractive characteristics of the eye. The procedure is taught by Refractec under the service marks CONDUCTIVE KERATOPLASTY and CK.

The current provided to the cornea changes the physiology of the stroma collagen tissue. The change in physiology varies the impedance of the tissue. The energy provided to the cornea by the Refractec console has a constant voltage. Consequently, the current provided to the cornea will vary with changes in the physiology and corresponding impedance of the cornea. It would be desirable to sense the changes in physiology and regulate the power provided to the cornea to optimize the results of a CK procedure.

### BRIEF SUMMARY OF THE INVENTION

An apparatus that provides energy to a probe placed in contact with a cornea to perform a medical procedure. The apparatus includes a circuit that delivers energy to the cornea through the probe, and a regulator circuit that controls the energy delivered to cornea during the medical procedure.

### BRIEF DESCRIPTION OF THE DRAWINGS

FIG. 1 is a perspective view of a thermokeratoplasty system;

FIG. 2 is a graph showing a waveform that is provided by a console of the system;

FIG. 3 is an enlarged view of a tip inserted into a cornea;

FIG. 4 is a top view showing a pattern of denatured areas of the cornea;

FIG. 5 is a schematic of a radio frequency electric circuit of the console;

FIG. 6 is a graph showing a voltage, a current and an impedance of a cornea during a CK procedure;

FIG. 7 is a graph similar to FIG. 6 showing a procedure where the tissue impedance did not rise;

FIG. 8 is a graph similar to FIG. 6 showing a procedure where the tissue impedance increased an undesirable amount.

### DETAILED DESCRIPTION

Disclosed is an apparatus that provides radio frequency energy to a probe in contact with a cornea to perform a medical procedure. The apparatus includes a radio frequency circuit that supplies RF energy to the probe and a regulator that regulates the delivery of RF energy during the medical procedure. The apparatus may include a sensing circuit that senses a change in a physiology of the cornea. The regulator can vary the RF energy delivered to the cornea in accordance with changes in the cornea physiology. For example, a waveform of tissue impedance may be determined and compared to a desired waveform. Deviations from the desired waveform may cause the regulator to increase or decrease the power applied to the cornea.

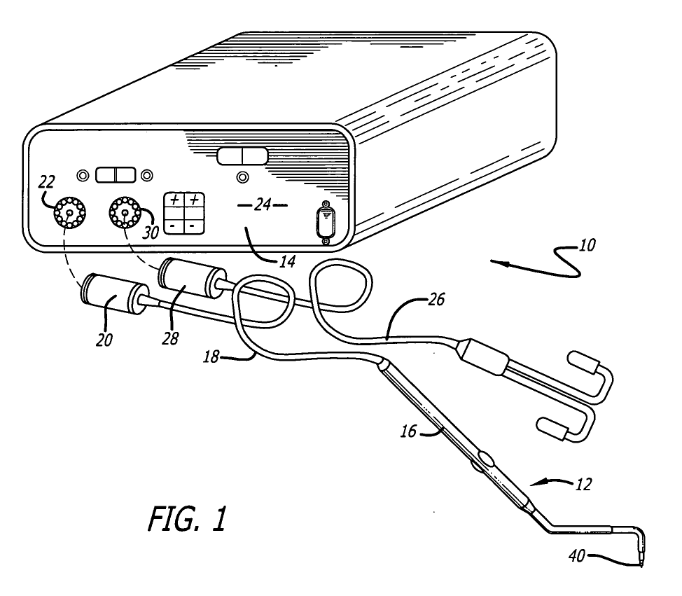

Referring to the drawings more particularly by reference numbers, FIG. 1 shows a thermokeratoplasty electrode system 10 of the present invention. The system 10 includes an electrode probe 12 coupled to a console 14. The console 14 contains a power supply that can deliver electrical power to the probe 12. The probe 12 has a hand piece 16 and wires 18 that couple the probe electrode to a connector 20 that plugs into a mating receptacle 22 located on the front panel 24 of the console 14. The hand piece 16 may be constructed from a non-conductive material.

The system 10 also includes a return element 26 that is in contact with the patient to provide a return path for the electrical current provided by the console 14 to the probe 12. The return element 26 has a connector 28 that plugs into a mating receptacle 30 located on the front panel 24 of the console 14. By way of example, the ground element may be a lid speculum that is used to maintain the patient's eyelids in an open position while providing a return path for the electrical current.

The console 14 provides a predetermined amount of energy, through a controlled application of power for a predetermined time duration. The console 14 may have manual controls that allow the user to select treatment parameters such as the power and time duration. The console 14 can also be constructed to provide an automated operation. The console 14 may have monitors and feedback systems for measuring physiologic tissue parameters such as tissue impedance, tissue temperature and other parameters, and adjust the output power of the radio frequency amplifier to accomplish the desired results.

In one embodiment, the console provides voltage limiting to prevent arcing. To protect the patient from overvoltage or overpower, the console 14 may have an upper voltage limit and/or upper power limit which terminates power to the probe when the output voltage or power of the unit exceeds a predetermined value.

The console 14 may also contain monitor and alarm circuits which monitors physiologic tissue parameters such as the resistance or impedance of the load and provides adjustments and/or an alarm when the resistance/impedance value exceeds and/or falls below predefined limits. The adjustment feature may change the voltage, current, and/or power delivered by the console such that the physiological parameter is maintained within a certain range. The alarm may provide either an audio and/or visual indication to the user that the resistance/impedance value has exceeded the outer predefined limits. Additionally, the unit may contain a ground fault indicator, and/or a tissue temperature monitor. The front panel 24 of the console 14 typically contains meters and displays that provide an indication of the power, frequency, etc., of the power delivered to the probe.

The console 14 may deliver a radiofrequency (RF) power output in a frequency range of 100 KHz-5 MHz. In the preferred embodiment, power is provided to the probe at a frequency in the range of 350 KHz. The console 14 is designed so that the power supplied to the probe 12 does not exceed a certain upper limit of up to several watts. Preferably the console is set to have an upper power limit of 1.2 watts (W). The time duration of each application of power to a particular corneal location can be up to several seconds but is typically set between 0.1-1.0 seconds. The unit 14 is preferably set to deliver approximately 0.6 W of power for 0.6 seconds.

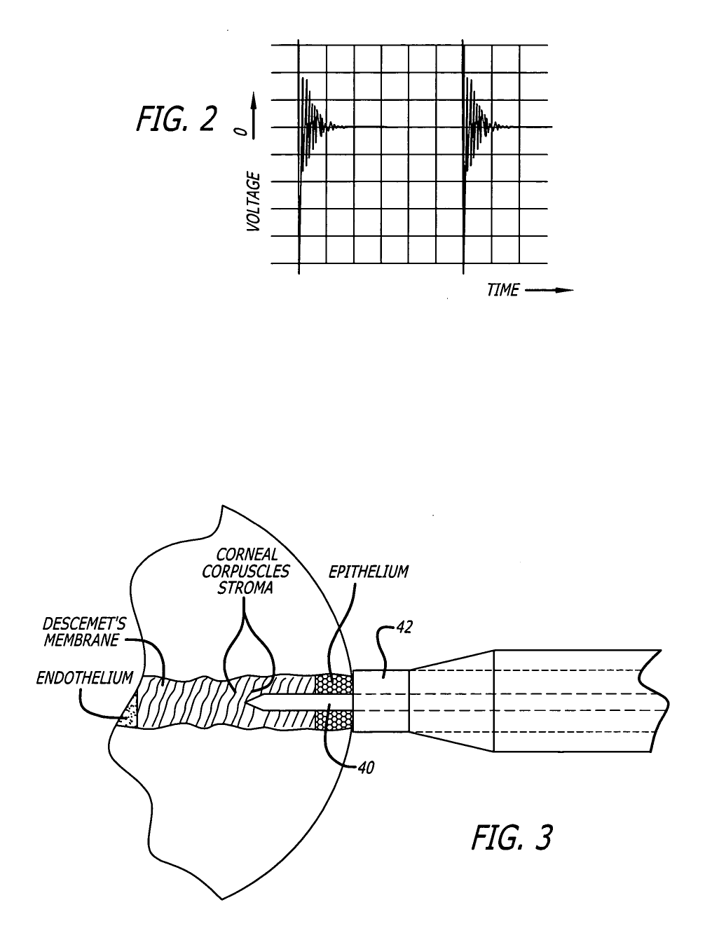

FIG. 2 shows a typical voltage waveform that is delivered by the probe 12 to the cornea. Each pulse of energy delivered by the probe 12 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

As shown in FIG. 3, during a procedure, an electrode tip 40 of the handpiece is inserted into a cornea. The length of the tip 40 is typically 300-600 microns, preferably 400 microns, so that the electrode enters the stroma layer of the cornea. The electrode may have a stop 42 that limits the penetration of the tip 40. The tip diameter is small to minimize the invasion of the eye.

The probe 12 provides a current to the cornea through the tip 40. The current denatures the collagen tissue of the stroma. Because the particular tip 40 is inserted into the stroma it has been found that a power no greater than 1.2 watts for a time duration no greater than 1.0 seconds will adequately denature the corneal tissue to provide optical correction of the eye. However, other power and time limits, in the range of several watts and seconds, respectively, can be used to effectively denature the corneal tissue. Inserting the tip 40 into the cornea provides improved repeatability over probes placed into contact with the surface of the cornea, by reducing the variances in the electrical characteristics of the epithelium and the outer surface of the cornea.

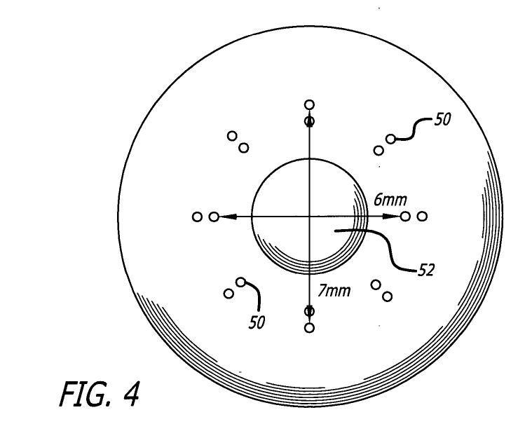

FIG. 4 shows a pattern of denatured areas 50 that have been found to correct hyperopic or presbyopic conditions. A circle of 8, 16, or 24 denatured areas 50 are created about the center of the cornea, outside the visual axis portion 52 of the eye. The visual axis has a nominal diameter of approximately 5 millimeters. It has been found that 16 denatured areas provide the most corneal shrinkage and less post-op astigmatism effects from the procedure. The circle of denatured areas typically have a diameter between 6-8 mm, with a preferred diameter of approximately 7 mm. If the first circle does not correct the eye deficiency, the same pattern may be repeated, or another pattern of 8 denatured areas may be created within a circle having a diameter of approximately 6.0-6.5 mm either in line or overlapping. The assignee of the present application provides instructional services to educate those performing such procedures under the service marks CONDUCTIVE KERATOPLASTY and CK.

The exact diameter of the pattern may vary from patient to patient, it being understood that the denatured spots should preferably be formed in the non-visionary portion 52 of the eye. Although a circular pattern is shown, it is to be understood that the denatured areas may be located in any location and in any pattern. In addition to correcting for hyperopia, the present invention may be used to correct astigmatic conditions. For correcting astigmatic conditions, the denatured areas are typically created at the end of the astigmatic flat axis. The present invention may also be used to correct procedures that have overcorrected for a myopic condition.

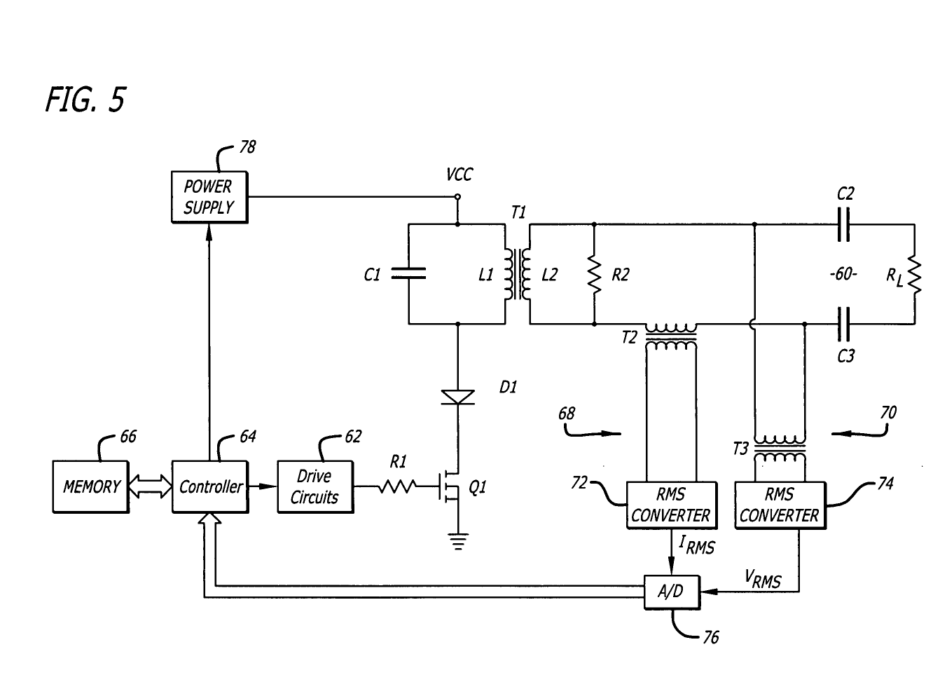

FIG. 5 shows an example of a console 14 that can apply RF power to a patient's cornea. The patient is represented by load resistor R_{L}. The console 14 may include a radio frequency circuit 60 that delivers RF energy to the cornea. The RF circuit 60 may include a transformer T1 that both stores and discharges energy. The transformer T1 has a primary winding L1 that is connected to a voltage supply line Vcc and to a switch Q1 by diode D1. The primary winding L1 is connected in parallel with capacitor C1.

The switch Q1 may be a MOSFET transistor with a gate coupled to a driver circuit 62 through resistor R1. The driver circuit 62 may be connected to a controller 64 that can turn the switch on and off. The circuit 60 may further have a pre-load resistor R2 and capacitors C2 and C3 connected to a secondary winding L2 of the transformer T1. The capacitors C2 and C3 filter undesirable low frequency current from flowing into the load R_{L}. The pre-load resistor R2 pulls some of the current from the winding through current division so that R_{L} limits over-voltage transients that may occur under open circuit conditions. The controller 64 may include a microprocessor that operates in accordance with operating instructions and data stored in memory 66. The memory 66 may include one or more memory devices, including volatile and non-volatile memory.

The console 14 includes a current sensing circuit 68 and a voltage sensing circuit 70 that sense the current and voltage delivered to the cornea, respectively. The current sensing circuit 68 senses the current flowing through the load R_{L}. The current sensing circuit 68 may include a transformer T2 in series with the load resistor R_{L}. The output current of the transformer T2 can be converted to an average rms value by an RMS converter 72. Likewise, the voltage sensing circuit 70 may have a transformer T3 that is in parallel with the load resistor R_{L}. The output voltage of transformer T3 can be converted to an average rms value by an RMS converter 74. The current rms, Irms, and voltage rms, Vrms, values can be converted into a digital format by an analog to digital converter 76. The digitized rms values can be provided to the controller 64. The controller 64 may multiply the rms values to determine the power provided to the cornea. As an alternate embodiment, the rms output signals provided by converters 72 and 74 can be multiplied in analog form and then converted to a digital format.

The controller 64 may regulate the power provided to the cornea based on the power sensed through the sensing circuits 68 and 70. The controller 64 may regulate the power about a single set-point. For example, the controller 64 may insure that 0.3 W of power is always provided to the cornea. The controller 64 may regulate power by changing the value of V_{CC} through the power supply 78, and/or by varying the time that that the transistor Q1 is turned on. The controller 64 may also regulate power in accordance with a set-point curve. For example, the set-point curve may be 0.2 W at t=0, 0.3 w at t=0.2 sec., 0.35 W at t=0.4 sec. and 0.4 W at t=0.6 sec. The controller 64 can receive the power feedback from the sensing circuits 68 and 70 and regulate the power to fit the set-point curve.

Although current and voltage sensing circuits are shown and described, it is to be understood that the console 14 may have just a current sensing circuit 68, or alternatively only a voltage sensing circuit 70. Additionally, the controller 64 may operate in a variety of different feedback controls such as proportional control, integral control, derivative control, or proportional integral derivative (PID) control theories.

In operation, the controller 64 turns the switch Q1 on which causes the primary winding L1 to store energy in the magnetic material of T1. The controller 64 turns off the switch Q1, wherein the magnetic material of the transformer T1 discharges its stored energy. The discharge creates a current in the primary loop with the capacitor C1. The frequency of the current is established by the capacitance and inductance values of the capacitor C1 and inductor L1, respectively. The current is also induced onto the secondary winding L2 and applied to the cornea. An example of the resultant waveform is shown FIG. 2. Although a damped waveform is shown and described, it is to be understood that other waveforms such as sinusoidal may be generated by the console 14. By way of example, a sinusoidal waveform can be produced by turning transistor Q1 on and off at a rate that is a function of the values for the passive elements of the radio frequency circuit 60.

The current sensing circuit 68 and voltage sensing circuit 70 provide feedback to the controller 64 to determine the power delivered by the probe to the cornea. If the power does not meet a predetermined criteria the controller 64 can change the voltage Vcc and/or the duration that the transistor Q1 is turned on during the next waveform cycle.

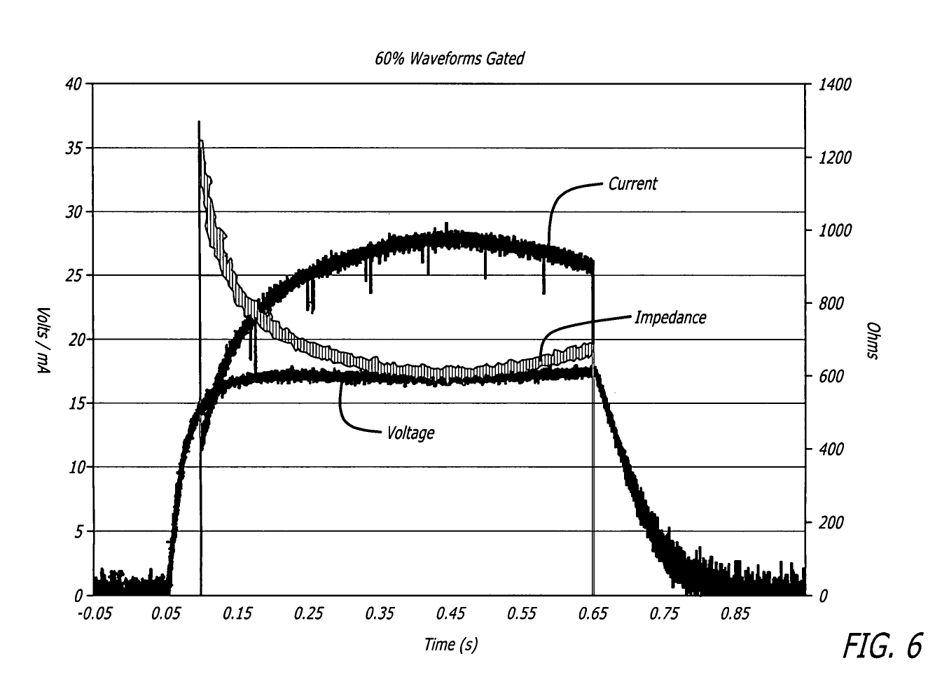

Application of current to a cornea will denature the cornea tissue and cause corresponding change in the ohmic value of the patient load resistance R_{L}. The current that flows through the load will change inversely with a variation in the resistance value of R_{L}. This is shown in FIG. 6, which shows the voltage, current and impedance at the patient load R_{L} during the application of power to a cornea. In a typical CK procedure the impedance of the load resistor R_{L} starts at approximately 1300 ohms and falls to approximately 600 ohms before slightly increasing again as shown in FIG. 6.

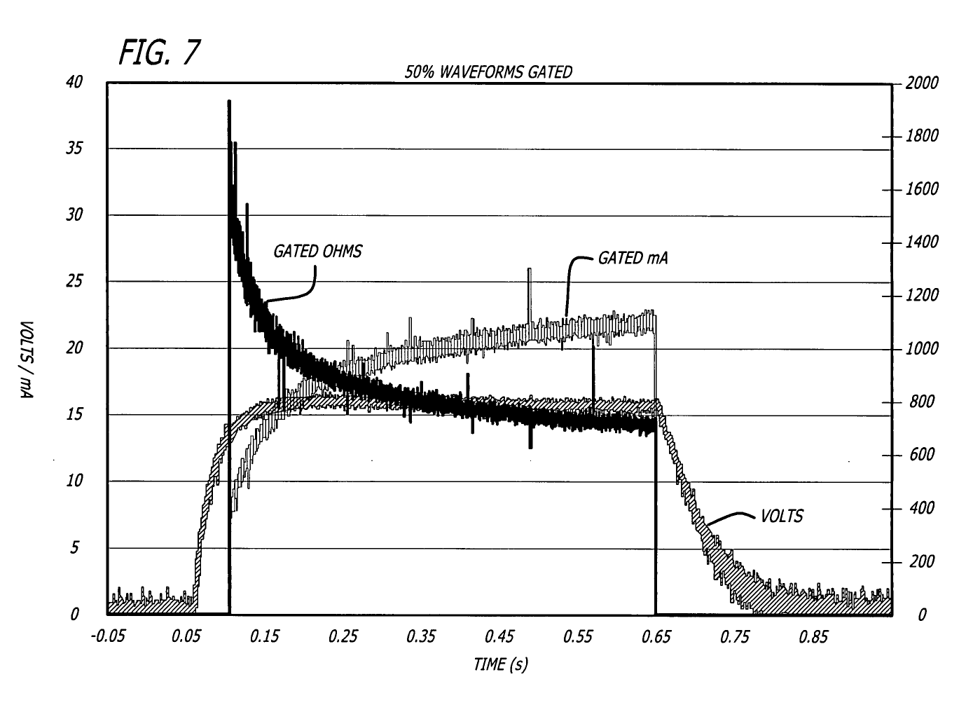

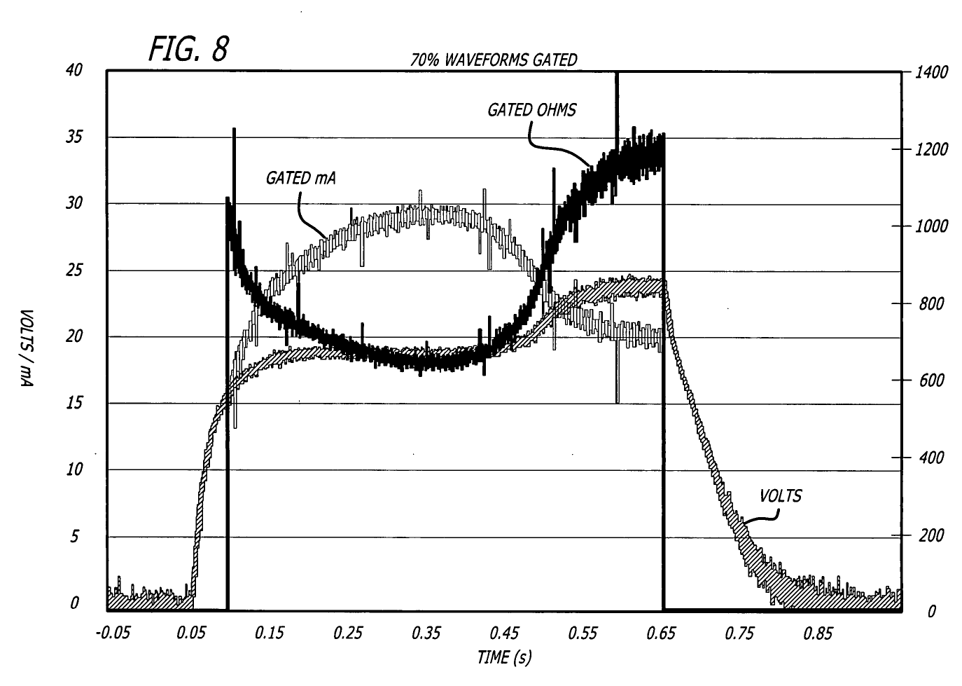

As shown in FIG. 7, the impedance may not rise during the application of RF energy. This is may be an indication that not enough energy was delivered to achieve a desired physiological result. Alternatively, as shown in FIG. 8, too much energy may be applied to the cornea, thereby creating a waveform that has a dramatic rise in impedance during the application of RF energy. This may be an indication of tissue necrosis.

The controller 64 can receive the feedback from the sensing circuits 68 and 70 and determine the impedance waveform. The controller 64 can then compare the impedance waveform with a desired waveform. The controller 64 can regulate the energy provided to the cornea in accordance deviations of the measured waveform from the desired waveform. For example, a measured waveform that fits the profile shown in FIG. 7, may cause the controller 64 to apply RF energy to the cornea for a longer time duration and/or increase the power level. The controller 64 may continually monitor the measured impedance and apply RF power until the impedance rises and matches the profile shown in FIG. 6. By way of example, the controller 64 may no longer provide energy to the cornea when the impedance increases by 5% or more. Alternatively, if the measured waveform fits the profile shown in FIG. 8, the controller may shorten the application or RF energy and/or lower the power level.

Although monitoring power or tissue impedance is described, the console may sense other parameters such as tissue temperature, tissue moisture content, etc. By way of example, the probe may have a temperature sensor or moisture sensor integrated into the tip. In general, the console 14 senses a physiological change in the corneal tissue and regulates the power delivered to the cornea to achieve a desired result. The controller 64 may create a desired waveform with upper and lower limits and adjust the power when a measured waveform exceeds the limits. The desired waveform, set-points etc. may be stored in memory 66 as a table and/or generated in accordance with an algorithm.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC), microwave, ultrasonic and light can be transferred into the cornea. Non-thermal energy does not include the concept of heating a tip that had been inserted or is to be inserted into the cornea.

By way of example, the circuit 60 can be modified to supply energy in the microwave frequency range or the ultrasonic frequency range. By way of example, the probe 12 may have a helical microwave antenna with a diameter suitable for corneal delivery. The delivery of microwave energy could be achieved with or without corneal penetration, depending on the design of the antenna. The system may modulate the microwave energy in response to changes in the characteristic impedance.

For ultrasonic application, the probe 12 would contain a transducer that is driven by the circuit 60 and mechanically oscillates the tip 40. The system could monitor acoustic impedance and provide a corresponding feedback/regulation scheme For application of light the probe may contain some type of light guide that is inserted into the cornea and directs light into corneal tissue. The console would have means to generate light, preferably a coherent light source such as a laser, that can be delivered by the probe. The probe may include lens, waveguide and a photodiode that is used sense reflected light and monitor variations in the index of refraction, birefringence index of the cornea tissue as a way to monitor physiological changes and regulate power.

1. An apparatus that provides a radio frequency energy to a probe placed in contact with a cornea to perform a medical procedure, comprising:
a radio frequency circuit that delivers a radio frequency energy to the cornea through the probe; and,
a regulator circuit that controls the radio frequency energy delivered to the cornea during the medical procedure.

2. The apparatus of claim 1, further comprising a sensing circuit that senses a change in a physiology of the cornea during the medical procedure and provides a feedback to said regulator circuit.

3. The apparatus of claim 2, wherein said sensing circuit senses a current delivered to the cornea.

4. The apparatus of claim 2, wherein said sensing circuit senses a voltage delivered to the cornea.

5. The apparatus of claim 2, wherein said sensing circuit senses a temperature of the cornea.

6. The apparatus of claim 2, wherein said sensing circuit senses an impedance of the cornea.

7. The apparatus of claim 2, wherein said sensing circuit senses a moisture of the cornea.

8. The apparatus of claim 1, wherein said regulator circuit controls the delivery of the radio frequency energy about a set-point.

9. The apparatus of claim 1, wherein said regulator circuit controls the delivery of the radio frequency energy about a set-curve.

10. The apparatus of claim 2, wherein said regulator circuit determines a profile of a physiological parameter and regulates the radio frequency energy delivered to the cornea in accordance with the profile.

11. The apparatus of claim 10, wherein the profile is an impedance profile.

12. The apparatus of claim 10, wherein the profile is a temperature profile.

13. The apparatus of claim 10, wherein the profile is a moisture profile.

14. The apparatus of claim 10, wherein said regulator circuit decreases the radio frequency energy if the profile includes an increase in impedance beyond a threshold level during the medical procedure.

15. The apparatus of claim 10, wherein said regulator circuit terminates delivery of the radio frequency energy if the profile exceeds a threshold level during the medical procedure.

16. The apparatus of claim 10, wherein said regulator circuit terminates delivery of the radio frequency energy if the profile includes a slope that exceeds a threshold level during the medical procedure.

17. The apparatus of claim 16, wherein said regulator circuit modulates a duration of the delivery of the radio frequency energy.

18. The apparatus of claim 17, wherein said duration is modulated in response to changes in a profile of the physiological parameter.

19. The apparatus of claim 18, wherein the physiological parameter is an impedance.

20. The apparatus of claim 18, wherein the physiological parameter is a temperature.

21. The apparatus of claim 18, wherein the physiological parameter is a tissue moisture.

22. The apparatus of claim 1, wherein said regulator circuit modulates a level of the radio frequency energy.

23. An apparatus that provides a radio frequency energy to a probe placed in contact with a cornea to perform a medical procedure, comprising:
a radio frequency circuit that delivers a radio frequency energy to the cornea through the probe; and,
regulator circuit means for controlling the radio frequency energy delivered to cornea during the medical procedure.

24. The apparatus of claim 23, further comprising sensing circuit means for sensing a change in a physiology of the cornea during the medical procedure and providing a feedback to said regulator circuit.

25. The apparatus of claim 24, wherein said sensing circuit means senses a current delivered to the cornea.

26. The apparatus of claim 24, wherein said sensing circuit means senses a voltage delivered to the cornea.

27. The apparatus of claim 24, wherein said sensing circuit means senses a temperature of the cornea.

28. The apparatus of claim 24, wherein said sensing circuit means senses an impedance of the cornea.

29. The apparatus of claim 24, wherein said sensing circuit means senses a moisture of the cornea.

30. The apparatus of claim 23, wherein said regulator circuit means controls the delivery of the radio frequency energy about a set-point.

31. The apparatus of claim 23, wherein said regulator circuit means controls the delivery of the radio frequency energy about a set-curve.

32. The apparatus of claim 24, wherein said regulator circuit means determines a profile of a physiological parameter and regulates the radio frequency energy delivered to the cornea in accordance with the profile.

33. The apparatus of claim 32, wherein the profile is an impedance profile.

34. The apparatus of claim 32, wherein the profile is a temperature profile.

35. The apparatus of claim 32, wherein the profile is a moisture profile.

36. The apparatus of claim 32, wherein said regulator circuit means decreases the radio frequency energy if the profile includes an increase in impedance beyond a threshold level during the medical procedure.

37. The apparatus of claim 32, wherein said regulator circuit means terminates delivery of the radio frequency energy if the profile exceeds a threshold level during the medical procedure.

38. The apparatus of claim 32, wherein said regulator circuit means terminates delivery of the radio frequency energy if the profile includes a slope that exceeds a threshold level during the medical procedure.

39. The apparatus of claim 23, wherein said regulator circuit means modulates a duration of the delivery of the radio frequency energy.

40. The apparatus of claim 39, wherein said duration is modulated in response to changes in a profile of the physiological parameter.

41. The apparatus of claim 40, wherein the physiological parameter is an impedance.

42. The apparatus of claim 40, wherein the physiological parameter is a temperature.

43. The apparatus of claim 40, wherein the physiological parameter is a tissue moisture.

44. The apparatus of claim 23, wherein said regulator circuit means modulates a level of the radio frequency energy.

45. A method for performing a medical procedure on a cornea, comprising:
placing a probe in contact with a cornea;
delivering a radio frequency energy to the cornea through the probe; and,
regulating the radio frequency energy delivered to cornea during the medical procedure.

46. The method of claim 45, further comprising sensing and feeding back a change in a physiology of the cornea during the medical procedure and regulating the radio frequency energy delivered to the cornea as a function of the feedback.

47. The method of claim 46, wherein a current delivered to the cornea is sensed during the medical procedure.

48. The method of claim 46, wherein a voltage delivered to the cornea is sensed during the medical procedure.

49. The method of claim 46, wherein a temperature of the cornea is sensed during the medical procedure.

50. The method of claim 46, wherein an impedance of the cornea is sensed during the medical procedure.

51. The method of claim 46, wherein said a moisture of the cornea is sensed during the medical procedure.

52. The method of claim 45, wherein the radio frequency energy is regulated about a set-point.

53. The method of claim 45, wherein the radio frequency energy is regulated about a set-curve.

54. The method of claim 46, wherein a profile of a physiological parameter is determined and the radio frequency energy delivered to the cornea is regulated in accordance with the profile.

55. The method of claim 54, wherein the profile is an impedance profile.

56. The method of claim 54, wherein the profile is a temperature profile.

57. The method of claim 54, wherein the profile is a moisture profile.

58. The method of claim 54, wherein regulating includes decreasing the radio frequency energy if the profile includes an increase in impedance beyond a threshold level during the medical procedure.

59. The method of claim 54, wherein regulating includes terminating delivery of the radio frequency energy if the profile exceeds a threshold level during the medical procedure.

60. The method of claim 54, wherein regulating includes terminating delivery of the radio frequency energy if the profile includes a slope that exceeds a threshold level during the medical procedure.

61. The method of claim 45, wherein regulating includes modulating a duration of the delivery of the radio frequency energy.

62. The method of claim 61, wherein the duration is modulated in response to changes in a profile of the physiological parameter.

63. The method of claim 61, wherein the physiological parameter is an impedance.

64. The method of claim 61, wherein the physiological parameter is a temperature.

65. The method of claim 61, wherein the physiological parameter is a tissue moisture.

66. The method of claim 45, wherein regulating includes modulating a level of the radio frequency energy.

67. An apparatus that provides a radio frequency energy to a probe placed in contact with a cornea to perform a medical procedure, comprising:
a radio frequency circuit that delivers a radio frequency energy to the cornea through the probe; and,
a sensing circuit that senses a change in a physiology of the cornea while said radio frequency circuit delivers the radio frequency energy to the cornea.

68. The apparatus of claim 67, wherein said sensing circuit senses a current delivered to the cornea.

69. The apparatus of claim 67, wherein said sensing circuit senses a voltage delivered to the cornea.

70. The apparatus of claim 67, wherein said sensing circuit senses a temperature of the cornea.

71. The apparatus of claim 67, wherein said sensing circuit senses an impedance of the cornea.

72. The apparatus of claim 67, wherein said sensing circuit senses a moisture of the cornea.

73. An apparatus that provides a radio frequency energy to a probe placed in contact with a cornea to perform a medical procedure, comprising:
a radio frequency circuit that delivers a radio frequency energy to the cornea through the probe; and,
sensing means for sensing a change in a physiology of the cornea while said radio frequency circuit delivers the radio frequency energy delivered to the cornea.

74. The apparatus of claim 73, wherein said sensing means senses a current delivered to the cornea.

75. The apparatus of claim 73, wherein said sensing means senses a voltage delivered to the cornea.

76. The apparatus of claim 73, wherein said sensing means senses a temperature of the cornea.

77. The apparatus of claim 73, wherein said sensing means senses an impedance of the cornea.

78. The apparatus of claim 73, wherein said sensing means senses an impedance of the cornea.

79. A method for performing a medical procedure on a cornea, comprising:
placing a probe in contact with a cornea;
delivering a radio frequency energy to the cornea through the probe; and,
sensing a change in a physiology of the cornea while the radio frequency energy is delivered to the cornea.

80. The method of claim 79, wherein a current delivered to the cornea is sensed while the radio frequency energy is delivered to the cornea.

81. The method of claim 79, wherein a voltage delivered to the cornea is sensed while the radio frequency energy is delivered to the cornea.

82. The method of claim 79, wherein an impedance of the cornea is sensed while the radio frequency energy is delivered to the cornea.

83. The method of claim 79, wherein a temperature of the cornea is sensed while the radio frequency energy is delivered to the cornea.

84. The method of claim 79, wherein a moisture of the cornea is sensed while the radio frequency energy is delivered to the cornea.

85. An apparatus that provides a non-thermal energy to a cornea through a probe to perform a medical procedure that denatures collagen tissue and reshapes the cornea, comprising:
an energy circuit that delivers a non-thermal energy to the cornea through the probe; and,
a regulator circuit that controls the non-thermal energy delivered to the cornea during the medical procedure.

86. The apparatus of claim 85, wherein the non-thermal energy is in a microwave frequency range.

87. The apparatus of claim 85, wherein the non-thermal energy is in an ultrasonic frequency range.

88. The apparatus of claim 85, wherein the non-thermal energy is light.

89. The apparatus of claim 85, wherein the non-thermal energy is direct current.

90. The apparatus of claim 85, further comprising a sensing circuit that senses a change in a physiology of the cornea during the medical procedure and provides a feedback to said regulator circuit.

91. The apparatus of claim 90, wherein said sensing circuit senses a current delivered to the cornea.

92. The apparatus of claim 90, wherein said sensing circuit senses a voltage delivered to the cornea.

93. The apparatus of claim 90, wherein said sensing circuit senses a temperature of the cornea.

94. The apparatus of claim 90, wherein said sensing circuit senses an impedance of the cornea.

95. The apparatus of claim 90, wherein said sensing circuit senses an optical characteristic of the cornea.

96. The apparatus of claim 85, wherein said regulator circuit controls the delivery of the non-thermal energy about a set-point.

97. The apparatus of claim 85, wherein said regulator circuit controls the delivery of the non-thermal energy about a set-curve.

98. The apparatus of claim 90, wherein said regulator circuit determines a profile of a physiological parameter and regulates the non-thermal energy delivered to the cornea in accordance with the profile.

99. The apparatus of claim 98, wherein said regulator circuit decreases the non-thermal energy if the profile displays changes indicative of necrotic collagen structural modification beyond a threshold level during the medical procedure.

100. The apparatus of claim 98, wherein said regulator circuit terminates delivery of the non-thermal energy if the profile exceeds a threshold level during the medical procedure.

101. The apparatus of claim 98, wherein said regulator circuit terminates delivery of the non-thermal energy if the profile includes a slope that exceeds a threshold level during the medical procedure.

102. The apparatus of claim 85, wherein said regulator circuit modulates a duration of the delivery of the non-thermal energy.

103. The apparatus of claim 85, wherein said regulator circuit modulates a level of the non-thermal energy.

104. An apparatus that provides a non-thermal energy to a cornea through a probe to perform a medical procedure that denatures collagen tissue and reshapes the cornea, comprising:
an energy circuit that delivers a non-thermal energy to the cornea through the probe; and,
regulator circuit means for controlling the non-thermal energy delivered to cornea during the medical procedure.

105. The apparatus of claim 104, wherein the non-thermal energy is in a microwave frequency range.

106. The apparatus of claim 104, wherein the non-thermal energy is in an ultrasonic frequency range.

107. The apparatus of claim 104, wherein the non-thermal energy is light.

108. The apparatus of claim 104, wherein the non-thermal energy is direct current.

109. The apparatus of claim 104, further comprising sensing circuit means for sensing a change in a physiology of the cornea during the medical procedure and providing a feedback to said regulator circuit.

110. The apparatus of claim 109, wherein said sensing circuit means senses a current delivered to the cornea.

111. The apparatus of claim 109, wherein said sensing circuit means senses a voltage delivered to the cornea.

112. The apparatus of claim 109, wherein said sensing circuit means senses a temperature of the cornea.

113. The apparatus of claim 109, wherein said sensing circuit means senses an impedance of the cornea.

114. The apparatus of claim 109, wherein said sensing circuit means senses an optical characteristic of the cornea.

115. The apparatus of claim 104, wherein said regulator circuit means controls the delivery of the non-thermal energy about a set-point.

116. The apparatus of claim 104, wherein said regulator circuit means controls the delivery of the non-thermal energy about a set-curve.

117. The apparatus of claim 109, wherein said regulator circuit means determines a profile of a physiological parameter and regulates the non-thermal energy delivered to the cornea in accordance with the profile.

118. The apparatus of claim 117, wherein said regulator circuit means decreases the non-thermal energy if the profile displays changes indicative of necrotic collagen structural modification beyond a threshold level during the medical procedure.

119. The apparatus of claim 114, wherein said regulator circuit means terminates delivery of the non-thermal energy if the profile exceeds a threshold level during the medical procedure.

120. The apparatus of claim 114, wherein said regulator circuit means terminates delivery of the non-thermal energy if the profile includes a slope that exceeds a threshold level during the medical procedure.

121. The apparatus of claim 104, wherein said regulator circuit means modulates a duration of the delivery of the non-thermal energy.

122. The apparatus of claim 104, wherein said regulator circuit modulates a level of the non-thermal energy.

123. A method for performing a medical procedure on a cornea, comprising:
contacting a cornea with a probe;
delivering a non-thermal energy to the cornea through the probe to denature collagen tissue and reshape the cornea; and,
regulating the non-thermal energy delivered to cornea during the medical procedure.

124. The method of claim 123, wherein the non-thermal energy is in a microwave frequency range.

125. The method of claim 123, wherein the non-thermal energy is in an ultrasonic frequency range.

126. The method of claim 123, wherein the non-thermal energy is light.

127. The method of claim 123, wherein the non-thermal energy is direct current.

128. The method of claim 123, further comprising sensing a change in a physiology of the cornea during the medical procedure and regulating the non-thermal energy delivered to the cornea as a function of the feedback.

129. The method of claim 128, wherein a current delivered to the cornea is sensed during the medical procedure.

130. The method of claim 128, wherein a voltage delivered to the cornea is sensed during the medical procedure.

131. The method of claim 128, wherein a temperature of the cornea is sensed during the medical procedure.

132. The method of claim 128, wherein an impedance of the cornea is sensed during the medical procedure.

133. The method of claim 128, wherein an optical characteristic of the cornea is sensed during the medical procedure.

134. The method of claim 123, wherein the non-thermal energy is regulated about a set-point.

135. The method of claim 123, wherein the non-thermal energy is regulated about a set-curve.

136. The method of claim 128, wherein a profile of a physiological parameter is determined and the non-thermal energy delivered to the cornea is regulated in accordance with the profile.

137. The method of claim 136, wherein regulating includes decreasing the non-thermal energy if the profile displays changes indicative of necrotic collagen structural modification beyond a threshold level during the medical procedure.

138. The method of claim 136, wherein regulating includes terminating delivery of the non-thermal energy if the profile exceeds a threshold level during the medical procedure.

139. The method of claim 136, wherein regulating includes terminating delivery of the non-thermal energy if the profile includes a slope that exceeds a threshold level during the medical procedure.

140. The method of claim 123, wherein regulating includes modulating a duration of the delivery of the non-thermal energy.

141. The method of claim 123, wherein regulating includes modulating a level of the non-thermal energy.

142. An apparatus that provides a non-thermal energy to a cornea through a probe to perform a medical procedure that denatures collagen tissue and reshapes the cornea, comprising:
a energy circuit that delivers a non-thermal energy to the cornea through the probe; and,
a sensing circuit that senses a change in a physiology of the cornea while said energy circuit delivers the non-thermal energy to the cornea.

143. The apparatus of claim 142, wherein the non-thermal energy is in a microwave frequency range.

144. The apparatus of claim 142, wherein the non-thermal energy is in an ultrasonic frequency range.

145. The apparatus of claim 142, wherein the non-thermal energy is light.

146. The apparatus of claim 142, wherein the non-thermal energy is direct current.

147. The apparatus of claim 142, wherein said sensing circuit senses a current delivered to the cornea.

148. The apparatus of claim 142, wherein said sensing circuit senses a voltage delivered to the cornea.

149. The apparatus of claim 142, wherein said sensing circuit senses a temperature of the cornea.

150. The apparatus of claim 142, wherein said sensing circuit senses an impedance of the cornea.

151. The apparatus of claim 142, wherein said sensing circuit senses an optical characteristic of the cornea.

152. An apparatus that provides a non-thermal energy to a cornea through a probe to perform a medical procedure to denature collagen tissue and reshape the cornea, comprising:
an energy circuit that delivers a non-thermal energy to the cornea through the probe; and,
sensing means for sensing a change in a physiology of the cornea while said energy circuit delivers the non-thermal energy delivered to the cornea.

153. The apparatus of claim 152, wherein the non-thermal energy is in a microwave frequency range.

154. The apparatus of claim 152, wherein the non-thermal energy is in an ultrasonic frequency range.

155. The apparatus of claim 152, wherein the non-thermal energy is light.

156. The apparatus of claim 152, wherein the non-thermal energy is direct current.

157. The apparatus of claim 152, wherein said sensing means senses a current delivered to the cornea.

158. The apparatus of claim 152, wherein said sensing means senses a voltage delivered to the cornea.

159. The apparatus of claim 152, wherein said sensing means senses a temperature of the cornea.

160. The apparatus of claim 152, wherein said sensing means senses an impedance of the cornea.

161. The apparatus of claim 152, wherein said sensing means senses an optical characteristic of the cornea.

162. A method for performing a medical procedure on a cornea, comprising:
contacting a cornea with a probe;
delivering a non-thermal energy to the cornea through the probe; and,
sensing a change in a physiology of the cornea while the non-thermal energy is delivered to the cornea.

163. The method of claim 162, wherein the non-thermal energy is in a microwave frequency range.

164. The method of claim 162, wherein the non-thermal energy is in an ultrasonic frequency range.

165. The method of claim 162, wherein the non-thermal energy is light.

166. The method of claim 162, wherein the non-thermal energy is direct current.

167. The method of claim 162, wherein a current delivered to the cornea is sensed while the non-thermal energy is delivered to the cornea.

168. The method of claim 162, wherein a voltage delivered to the cornea is sensed while the non-thermal energy is delivered to the cornea.

169. The method of claim 162, wherein a temperature of the cornea is sensed while the non-thermal energy is delivered to the cornea.

170. The method of claim 162, wherein an optical characteristic of the cornea is sensed while the non-thermal energy is delivered to the cornea.
